(19) United States
(12) Patent Application Publication
(10) Pub. No.: US 2021/0137438 A1
(43) Pub. Date: May 13, 2021

(54) CONTROL SYSTEM FOR MOBILE ROBOTS

(71) Applicant: Hewlett-Packard Development Company, L.P., Spring, TX (US)

(72) Inventors: Jonathan Salfity, Palo Alto, CA (US); Will Allen, Corvallis, OR (US); Hiroshi Horii, Palo Alto, CA (US)

(73) Assignee: Hewlett-Packard Development Company, L.P., Spring, TX (US)

(21) Appl. No.: 16/629,848

(22) PCT Filed: Oct. 31, 2017
(86) PCT No.: PCT/US2017/059201

§ 371 Date: Jan. 9, 2020

(51) Int. Cl. A61B 5/16; G05D 1/02; B25J 19/02; G06K 9/00

(52) U.S. Cl. CPC A61B 5/165; G06K 9/00302; B25J 19/023; G05D 1/0246

(57) ABSTRACT

Example implementations relate to control systems for mobile robots. An example control system obtains sensor data from multiple onboard sensors of a robot, and, based at least in part on input detected from the multiple on-board sensors, the control system determines a physical characteristic of a human actor moving in a region where the robot operates. Based on the physical characteristic, the control system may detect a mood of the human actor and alter the operation of the robot in response to the detected mood.

### BACKGROUND

Mobile robot technology has advanced to the point where robots operate among humans to perform tasks (e.g., delivery of items, operation of vehicles). Robots typically deploy sensors for detecting the presence of humans and other objects in the environment in order to perform their tasks and adjust the manner in which the robot operates.

### BRIEF DESCRIPTION OF THE DRAWINGS

FIG. 1A illustrates an example robot that is responsive to a detected mood of a human actor.

FIG. 1B illustrates an example robot, such as described by FIG. 1A, operating in a shared environment.

FIG. 1C illustrates another example robot, such as described by FIG. 1A, operating in a shared environment.

FIG. 1D illustrates another example robot, such as described by FIG. 1A, that alters its operation in response to detecting a mood of a human actor.

FIG. 1E illustrates another example robot, such as described by FIG. 1A, that uses an offboard sensor to determine a mood of a human actor.

FIG. 2 illustrates an example control system for use with a robot.

FIG. 3 illustrates an example method to control a robot to detect and respond to a mood of human actor.

### DETAILED DESCRIPTION

An example control system is provided to select or alter an action of a robot in response to detected movements of human actors to increase the levels of safety and enjoyment experienced by the human actors in a shared environment (i.e., an environment in which robots operate where humans are also present). As described with various examples, one or more processors of the control system execute instructions to obtain sensor data from multiple onboard sensors of the robot. Based at least in part on input detected from the onboard sensors, the processor determines a physical characteristic of a human actor that is static or moving in a region where the robot operates, and, based on the detected mood, causes the robot to alter its operation.

As used herein, a human actor can include a single human, multiple humans in a group, or a human-operated mechanism.

Some examples described herein can require the use of computing devices, including processing and memory resources. For example, one or more examples described herein may be implemented, in whole or in part, on computing devices such as servers, desktop computers, cellular or smartphones, and tablet devices. Memory, processing, and network resources may be used in connection with the establishment, use, or performance of any example described herein (including the performance of any method or in the implementation of any system).

Furthermore, one or more examples described herein may be implemented through the use of instructions that are executable by one or more processors. These instructions may be carried on a computer-readable medium. Machines shown or described with figures below provide examples of processing resources and computer-readable media on which instructions for implementing examples described herein can be carried and/or executed. In particular, the numerous machines shown with examples described herein include processors and various forms of memory for storing data and instructions. Examples of computer-readable media include permanent memory storage devices, such as hard drives on personal computers or servers. Other examples of computer storage media include portable storage units, such as CD or DVD units, flash memory (such as carried on smartphones, multifunctional devices or tablets), and magnetic memory. Computers, terminals, network enabled devices (e.g., mobile devices, such as cell phones) are all examples of machines and devices that utilize processors, memory, and instructions stored on computer-readable media.

Among other advantages, examples recognize that robots continue to be steadily integrated into everyday environments, including the home and workplace. However, while mobile robots are more prevalent than ever, human actors are still developing the instincts and expectations that are required to safely and cordially share an environment with mobile robots. Accordingly, examples are described in which a control system for mobile robots is able to recognize and act on “human” aspects of human behavior. Specifically, examples recognize a mood of a human actor, where the mood may reflect an emotional state of the human. According to examples, a robot may use sensor data to determine the mood of the human actor as one of a predefined set of emotional states. In variations, an example robot may detect a magnitude or strength of a human actor's emotional state. As used herein, the term “mood” is meant to reflect an emotional state of a human by type, and in some cases, by type and magnitude or strength.

FIG. 1A illustrates an example robot that is responsive to a detected mood of a human actor. A robot 100, such as shown by an example of FIG. 1A, can include any machine capable of carrying out a complex series of actions automatically. The robot 100 may be designed or constructed to take on any particular form (e.g., humanoid, TurtleBot, etc.). Examples may include any type of mobile robot configured to interact with a human actor in a shared environment. For example, the robot 100 may include an autonomous robot configured to interact and communicate with humans or other autonomous physical agents based on social behaviors or a particular role assigned to the robot (e.g., social robot). In other examples, the robot 100 may include a robot that performs behaviors or tasks autonomously (e.g., autonomous robots) where the degree of autonomy may depend upon the environmental context in which the robot is to perform its behaviors or tasks (e.g., domestic, industrial, etc.).

In an example of FIG. 1A, the robot 100 includes a control system 130 that controls a propulsion mechanism 140 in a manner that enables the robot 100 to dynamically operate in a shared environment, or region 101, such as a dwelling, office, sidewalk, roadway, etc. The control system 130 can include a memory 110 (e.g., a read-only memory (ROM) or random access memory (RAM)) that stores a set of mood detection instructions 112. The control system 130 may also include one or more processors 120 that execute the instructions stored in the memory 110.

As shown by an example of FIG. 1, the control system 130 may be integrated with the robot 100. For example, the control system 130 may be implemented using hardware, firmware and/or software. In variations, the control system 130 may be implemented using logic and/or processing resources which are remote from the robot 100. For example, the control system 130 can be implemented using a server, or a combination of servers, which communicate with components of the robot 100 (or fleet of robots) using a wireless network channel. In this way, the robot 100 (or each individual robot in a fleet) may act as a node to receive commands from the control system 130 while reporting back environmental information to the control system 130.

The propulsion mechanism 140 can be implemented using, for example, a motor and steering mechanism, to enable the robot to be accelerated and guided in multiple directions.

The robot 100 can include multiple onboard sensors for facilitating the detection and processing of objects in general, and more specifically, of human actors, as well as of a predetermined set of physical characteristics of human actors. Examples of sensors include a distance sensor 132, one or multiple types of image sensors 134, a wireless receiver 136, and a haptic sensor 138, among others.

The distance sensor 132 may be any type of sensor that can measure a distance from the robot 100 to a target object or actor in the region 101. The distance sensor 132 may employ both active distance measuring methods (e.g., laser-based, radar, LiDAR, ultrasonic, etc.) and/or passive distance measuring methods (e.g., detecting the noise or radiation signature of an object). In variations, a pair of stereoscopic cameras can be used to capture image and distance information. In addition to determining a distance between an object in the region 101 and the robot 100, the control system 130 may collect and process sensor data from the distance sensor 132 over an interval of time to calculate various other attributes of the human actor in the region 101. For example, the control system 130 may determine a velocity of a human actor in the region 101 by comparing the distance traveled by the actor over the interval of time. In some examples, the robot 100 includes multiple distance sensors 132, and the control system 130 can combine sensor data from them to develop more accurate estimates of the depth of objects located relative to the robot 100 in the region 101.

The image sensor(s) 134 may include any sensor that can acquire still images and/or video from the region 101 where the robot 100 operates. For example, the image sensor can include visible-light cameras, stereoscopic camera, etc. In addition, the control system 130 can utilize algorithms to extract information from the digital images and videos captured by the image sensor 134 to gain a high-level understanding of those digital images or videos (e.g., computer vision algorithms). In addition, the robot 100 can include multiple image sensors, from which sensor data may be combined to develop more accurate and precise determinations of the physical characteristics of the human actors in the region 101.

The haptic sensor(s) 138 may include any sensor that measures information that may arise from a physical interaction between the robot 100 and a human actor(s) or their shared environment. For example, a human actor may interact with the robot 101 by touching or petting the robot 101.

The wireless receiver 136 may include a sensor or radio to receive data from offboard sensors distributed in the region 101. For example, an offboard image sensor (e.g., a surveillance camera located in a building where the robot 100 operates) may publish or otherwise make available a video data stream. The wireless receiver 136 may receive (i.e., over a wireless network such as Bluetooth) the video data stream in addition to metadata associated with the video data stream, such as a time of the video and the physical coordinates of the offboard image sensor recording the video. In another example, the wireless image sensor 136 receives data from an offboard motion detecting device (e.g., occupancy sensor), or network of motion detecting devices, located within the region 101. In this way, the robot 100 “borrows” the perspective or field of view of an offboard sensor, which allows the control system 130 access to image data even when the perspective or field of view of the robot 100 is obscured or otherwise blocked. In some variations, the control system 130 is able to operate in a mode in which the robot 100 receives (e.g., via the wireless receiver 136) sensor data from multiple offboard sensors, from which the robot 100 can determine moods of persons, and determine appropriate response actions based on the determined moods. In such variations, the robot 100 can rely or otherwise incorporate an alternative perspective captured from the point of view of remote sensing devices.

Still further, in some variations, the control system 130 may acquire sensor data from offboard sensing devices that are provided with a mobile computing device of a user. For example, a smartphone or feature phone (or tablet, wearable device, or any other device worn or held closely by a human actor in or around the region 101) can acquire a variety of different types of sensing information about the user, including sensing information about the movement (e.g., a velocity or gait of the user's walk) or other physical characteristic of the user.

These examples and others with regard to distance sensor 132, image sensor 134, and wireless image sensor 136 are discussed in more detail below in the context of FIG. 1B through FIG. 1E.

According to the example of FIG. 1A, the control system 130 stores mood detection instructions 112 in memory 110. The mood detection instructions 112 may be executable by the processor 120 of the control system 130, to cause the robot 100 to determine a set of physical characteristics of a human actor that is detected from the sensor data 111, and to determine a mood of the human actor based on the determined set of physical characteristics. As described in greater detail, the control system 130 may signal a response 115 to the propulsion mechanism 140 upon making the mood determination. In this way, the control system 130 is able to process sensor data 111 to determine a mood of a human actor, and further to respond to the determined mood of the human actor.

In some aspects, the set of physical characteristics that are detected by the control system 130 can include a physical gesture, a facial expression, a characteristic of movement, a posture or orientation, or other characteristic that is detectable by the multiple onboard sensors. For example, physical characteristics can be determined for eye movement or orientation, head orientation, lip features (e.g., length, shape, etc.), posture, arm position, and other features. As an addition or alternative, the physical characteristics which are detected from the human actor may relate to characteristics of the human actor's movement. For example, the velocity, movement pattern (e.g., walking gate), linearity or other characteristic of movement can be detected from performing image analysis on the human actor for a duration of time (e.g., 1 second). In such examples, the physical characteristics may be detected in context of other information, such as whether the user is moving and how fast the user is moving.

Based on the detected physical characteristics, the control system 130 can determine a mood of the human actor. The control system 130 may, for example, map the detected physical characteristics to a mood or mood value. Alternatively, the control system 130 may use one or more models to determine the mood or mood value of the human actor. The determination of the mood may be either determinative (so that one mood is assumed) or probabilistic (so that multiple possible moods may be detected and weighted). The response to the human and the detected mood may include (i) maintain existing state (i.e., take no additional action other than the one the robot is performing), (ii) adjust or change an existing action that the robot is performing (e.g., robot moves slower), and/or (iii) perform a new action. Based on the determined mood of the human actor, the control system 130 can select a response to control the robot 100. In some examples, the selection of the response may be based on the determined mood of the human, as well as the existing action or operation of the robot. For example, if the human actor is detected to be “rushed,” the response from the robot may be to move aside if the robot is moving towards the human actor. If the robot is still, another response may be selected (e.g., stay still, turn on light, etc.). Still further, in some examples, the response to the detected mood may be based on other information, such as whether the human is moving, the direction the human is moving, contextual information (e.g., weather, lighting, surrounding, etc.).

In some examples, the control system 130 utilizes mood determination in the context of operating the robot to treat humans as dynamic objects that are to be detected and avoided. For example, the robot may implement a default response when humans are encountered. The mood determination, however, may change or influence the robot response that would have otherwise resulted from a non-human object. By determining the mood of human actors, the control system 130 can adjust the operation of the robot 100 to increase efficiency, safety, and the enjoyment of human actors sharing an environment with the robot.

With reference to examples of FIG. 1B and FIG. 1C, the robot 100 and the human actor both share and operate in a region 101. The control system 130 of the robot 100 may determine that a physical characteristic, such as a gaze of the human actor, is directed away from the robot 100 (e.g., the human actor may be looking at a watch, phone, or other person). Based on the gaze of the human actor, the control system 130 may detect that the human actor is distracted and either halt the operation of the robot 100 or alter its operation to move out of the path of the human actor to avoid an unsafe interaction (e.g., the human actor tripping over the robot 100).

In other examples, the physical characteristic of the human actor can include a relative or absolute velocity of the human actor based on a combination of data sensed from the distance sensor 132 and the image sensor 134. Based on the velocity of the human actor, the control system 130 may detect that the human actor is in a hurry and alter its behavior to avoid an unsafe interaction.

In addition to causing the robot 100 to alter its operation to avoid unsafe interactions with human actors in a shared environment, the control system 130 can cause the robot 100 to alter its operation to engage or initiate encounters with human actors based on determined physical characteristics of the human actors. For example, based on processing data from the robot 100 sensors, the control system 130 may detect that multiple human actors are looking at one another and not moving. In addition, the control system 130 may determine one or more facial landmarks, such as a smile on the face of one of the multiple human actors. Based on detecting these physical characteristics, the control system 130 may determine that the multiple human actors are engaged in a friendly conversation. In such an example, the control system 130 may alter the operation of the robot 100 to cause the robot 100 to approach the multiple human actors and socially interact with the multiple human actors (e.g., tell a joke).

Examples also provide for utilizing offboard sensors for detecting the mood of a human actor. According to the example of FIG. 1D, the robot 100 operates in the region 101 that is shared with a human actor. Examples recognize that in certain situations, the human actor may not be detectable by the onboard distance sensor 132. For example, the human actor may be outside of the field of view of the onboard image sensor 134 of the robot 100. In some examples, the control system 130 may also be communicatively linked to remote sensors such as the wireless image sensor 136, to process a video stream and its associated metadata from an offboard image sensor 152 (e.g., surveillance camera). The control system 130 may access and retrieve real-time video data from the wireless image sensor 136 as a response to detecting, for example, the presence of blind spots in the region 101. As an alternative or variation, the control system 130 may continuously or repeatedly retrieve the video data from remote image sensors when the robot 100 is sufficiently proximate to receive such data. The control system 130 can process the video stream data and its associated metadata from the wireless image sensor 136 in order to detect objects in the region 101 that may be in blind spots relative to the image sensor 134. The control system 130 may analyze image data from the wireless image sensor 136 in a similar fashion as the onboard sensors in order to determine a set of physical characteristics for the human actor which are indicative of the actor's mood. With reference to an example of FIG. 1D, the control system 130 may receive the video stream and its associated metadata to determine the velocity and likely path of the human actor moving through the region 101. Based on the velocity and path of the human actor, the control system 130 may determine that, if the robot 100 continues traveling on its current path at its current rate, a potential collision may occur between the robot 100 and the human actor. As a result, the control system 130 may cause the robot 100 to stop or alter its course of operation to avoid a potential collision.

FIG. 1E provides a further example of the robot 100 utilizing an offboard sensor to alter the operation of the robot 100. The control system 130 of robot 100 may receive, via the wireless image sensor 136, a signal from an offboard occupancy sensor 154 that determines the number of people in the region 101. Based on the signal from the offboard occupancy sensor 154, the control system 130 may determine that the region 101 includes more human actors than typically occupy the region 101 and, as a result, the control system 130 may cause the robot 100 to reduce its velocity or navigate to the perimeter of the region 101. In this way, the control system 130 detects a “mood” of the human actors in the region 101 in that the control system 130 determines a physical characteristic of the human actors in the region (e.g., absolute number of human actors in the region 101) and detects a pattern or change in a pattern (e.g., more than usual number of human actors in the region 101). The detected physical characteristics as pertaining to patterns may also relate to time. For example, a region such as a cafeteria in a workspace environment may not experience a particularly heavy flow of traffic in the morning hours, leaving a robot to operate freely. However, the control system may also utilize contextual information to make an alternative understanding of some detected characteristics. For example, the control system 130 may anticipate that, during lunch hours, the flow of traffic may increase, and consequently the control system 130 may cause the robot 100 to alter its operation to blend in with the increased flow of traffic.

The control system 130 may also include a feedback mechanism to modify the behavior of the robot in response to a detected mood. The feedback mechanism may be in the form of a sensor set (e.g., image sensors, offboard sensors) which captures, for example, physical characteristics of the human after the interaction with the robot. After the interaction, the feedback mechanism may capture, for example, an outcome between human and robot, or a second mood determination for the human. The data sets corresponding to such outcomes and determinations of the feedback mechanism may be used to further train the robots 100, and to tune the behavior of the robots over time. As described with some examples, the robot's behavior may be developed using models that can be trained on feedback that correlate to monitoring (e.g., using image sensors of the robot 100 or offboard sensors) an action of a human and comparing the detected action with what was predicted for the human based on the developed models. The feedback mechanism may include processing a large amount of recorded data related to interactions between the robot 100 and human actor(s) along with the respective outcomes for each interaction in order to develop and implement a more advanced and improved policy or behavior for the control system 130 to implement (e.g., deep reinforcement learning). For example, in reference to the example of FIG. 1B above in which the control system 130 causes the robot 100 to alter its operation to move from the path of a distracted human actor, the control system 130 may cause the robot 100 to veer only slightly from its path, resulting in a collision or near collision between the robot 100 and the human actor. The control system 130 may detect the near-miss event (e.g., using image data) and record the result of the robot's response as feedback for developing the model used to determine the robot's response. With additional tuning or training of the robot's response model, the model may communicate a different response to the next incident where a distracted human actor is detected. For such instances, the model may modify the instructions to the robot 100 to veer an even greater distance from the path of the distracted human actor upon the next such encounter. In addition, the learned behaviors or policies developed and implemented by one robot may be shared with other robots in the same network or environment.

FIG. 2 illustrates an example control system for use with a robot. A control system 200, such as described by an example of FIG. 2, can be implemented with a robot, such as described by examples of FIG. 1A through FIG. 1E using, for example, components described with an example of FIG. 1A through FIG. 1E.

In FIG. 2, the control system 200 includes a processor 210 and a memory 220. The memory 220 can be of any form, including RAM, DRAM or ROM. The memory 220 can store instructions, such as through installation of software (e.g., an application). The processor 210 may access instructions from the memory 220 to control the robot 100. According to some examples, the processor 210 accesses multiple sets of instructions, including: (i) a first set of instructions 212 to obtain sensor data from multiple sensors (e.g., onboard sensors, external sensors); (ii) a second set of instructions 214 to detect physical characteristics of a human actor from the sensor data; (iii) a third set of instructions 216 to detect a mood of a human actor using the determined physical characteristics; and (iv) a fourth set of instructions 218 to cause a robot to alter its operations based on the detected mood of the human actor. According to some examples, the processor 210 may access the instructions from the memory 220 to cause the robot 100 to perform a series of operations, as described in greater detail with an example of FIG. 3.

FIG. 3 illustrates an example method for causing a robot to alter its operation based on a detected mood of a human actor. The example method of FIG. 3 can be implemented using components illustrated in FIG. 1A through FIG. 1E and FIG. 2. Accordingly, references made to elements of FIG. 1A through FIG. 1E and FIG. 2 are for the purposes of illustrating a suitable element or component for performing an element of the example being described.

A processor of a computer system obtains sensor data from multiple onboard sensors of a robot (310). The sensor data can include input detected from onboard distance sensors 132 or onboard image sensors 134. In the examples above, the onboard distance sensors 132 and the onboard image sensors 134 are attached or otherwise affixed to the robot. As such, with reference to FIGS. 1A, 1B and 1C, the onboard distance sensors 132 and the onboard image sensors 134 obtain sensor data from the perspective or field of view of the robot as it traverses through the region 101.

In variations, sensor data may be obtained from a perspective or field of view other than that of the robot. For example, in reference to FIG. 1D, an offboard image sensor 152 (e.g., a surveillance camera) obtains sensor data from the perspective or field of view of the offboard image sensor 152. In the example of FIG. 1E, the perspective or field of view may be that of multiple offboard occupancy sensors that generate data which, when combined, may be used to determine a more accurate and more efficient calculation as to the number of human actors in a region. In the examples of FIG. 1D and FIG. 1E, the data generated from the offboard sensors (e.g., offboard image sensor, offboard occupancy sensor, etc.) may be reported out or transmitted to the wireless image sensor 136 of the robot to be processed by the robot or a control system of the robot. In this way, when the robot finds itself in a potentially disadvantageous position (e.g., turning a corner), the robot may “borrow” sensor data from offboard sensors to gain the perspective or field of view that would not otherwise be available to the robot. Still further, in some variations, the robot may utilize a network of sensors which may reside on the robot and/or outside of the robot. The robot may access and use sensor information from any of multiple types of sensors in the sensor network in order to determine information relative to mood determination of individual persons the robot encounters. Thus, the robot may utilize off-board sensors exclusively, or in combination with onboard sensors, and further utilize a variety of different types of sensors as it traverses a given region.

Based at least in part on input detected from the multiple onboard sensors, the processor of the computer system determines a physical characteristic of a human actor that is located in the same region where the robot operates (320). The human actor can be static or dynamic. For example, as discussed above, the computer system can detect several attributes of a dynamic human actor in the region (e.g., velocity, predicted path, gestures, etc.). However, the computer system can also detect several attributes of a static human actor in the region. In reference to the examples of FIG. 1B and FIG. 1C discussed above, the robot may encounter multiple human actors not moving. Further, multiple onboard sensors may detect facial landmarks to determine that the multiple human actors are gazing at one another. In addition, the static and dynamic physical characteristics of the human actor(s) in the shared region where the robot operates may be detected by the multiple onboard sensors of the robot or may be detected by an offboard sensor and then reported or transmitted to the robot or computer system to be processed as if the dynamic or static physical characteristic had been detected by an onboard sensor of the robot (e.g., onboard distance sensor, onboard images sensor, etc.).

Based on the physical characteristics determined, the processor of the computer system can detect a mood of the human actor (330) and then cause the robot to alter its operation accordingly (340). The detected mood of the human actor can include various types of emotions, behaviors, patterns of behaviors, etc. For example, when the computer system determines that a physical characteristic, such as the velocity of the human actor, is greater than the normal velocity at which other human actors in the region are traveling or usually travel, then the computer system may detect that the human actor is in a hurry and alter its operation to either move from the path of the human actor or, if the computer system initially set out to cause the robot to interact with the human actor, to instead not interact with the human actor.

In other examples, for multiple human actors determined to be engaged in conversation, the computer system may process the facial landmarks detected by the multiple sensors of the robot and process the facial landmarks with a computer vision algorithm to determine that the multiple human actors are in a “friendly” mood. As such, based on the friendly mood of the multiple human actors, the computer system may alter the operation of the robot to interact with the multiple human actors (e.g., say hello, tell a joke, etc.) whereas the robot would otherwise continue through the region without interacting with the multiple human actors.

In this way, the examples discussed here, and other variations extending therefrom, provide for robots that can interact in a shared environment with human actors, and, using rich sensing and processing capabilities, alter the operation of a robot to create a safer and even more amicable shared environment between the robot and human actors operating in the shared environment.

It is contemplated for examples described herein to extend to individual elements and concepts described herein, independently of other concepts, ideas or systems, as well as for examples to include combinations of elements recited anywhere in this application. Although examples are described in detail herein with reference to the accompanying drawings, it is to be understood that the concepts are not limited to those precise examples. Accordingly, it is intended that the scope of the concepts be defined by the following claims and their equivalents. Furthermore, it is contemplated that a particular feature described either individually or as part of an example can be combined with other individually described features, or parts of other examples, even if the other features and examples make no mention of the particular feature. Thus, the absence of describing combinations should not preclude having rights to such combinations.

What is claimed is:

1. A control system for a robot, the control system comprising:
a memory to store instructions;
at least one processor to execute the instructions to:
obtain sensor data from multiple sensors that are aligned to sense a region where the robot operates;
determine, based on input detected from the multiple sensors, a physical characteristic of a human actor that is located in the region where the robot operates;
detect a mood of the human actor based on the physical characteristic; and
based on the detected mood of the human actor, cause the robot to alter its operation.

2. The control system of claim 1, further comprising: a camera, and wherein the at least one processor executes the instructions to determine the physical characteristic by using the camera to detect a physical facial characteristic of the human actor.

3. The control system of claim 1, wherein at least one of the multiple sensors is offboard from the robot.

4. The control system of claim 1, wherein the at least one processor executes the instructions to detect the mood of the human actor by detecting, via a wireless receiver communicating with an offboard sensor, a location of the human actor, the location not within a field of view of an onboard camera sensor provided on the robot.

5. The control system of claim 4, wherein the offboard sensor includes a mobile computing device of the human actor.

6. The control system of claim 5, wherein the mobile computing device of the human actor communicates a velocity of the human actor.

7. The control system of claim 1, wherein the at least one processor executes the instructions to detect the mood of the human actor by detecting, based on input detected from the multiple sensors, a traffic pattern in the region where the robot operates.

8. The control system of claim 1, further comprising:
a feedback mechanism to modify the operation of the robot in response to the detected mood.

9. A robot comprising:
multiple onboard sensors;
a propulsion mechanism;
at least one processor coupled to the multiple onboard sensors;
a memory coupled to at least one processor, the memory storing instructions that, when executed by the at least one processor, cause the robot to perform operations that include:
obtaining sensor data from the multiple onboard sensors;
determining, based on input detected from the multiple onboard sensors, a physical characteristic of a human actor that is located in a region where the robot operates;
detecting a mood of the human actor based on the physical characteristic;
selecting a response to the detected mood; and
controlling the propulsion mechanism to implement the response.

10. The robot of claim 9, further comprising: a camera, and wherein determining the physical characteristic includes using the camera to detect a physical facial characteristic of the human actor.

11. The robot of claim 9, wherein detecting the mood of the human actor includes detecting, via a wireless receiver communicating with an offboard sensor, a location of the human actor, the location not within a field of view of an onboard camera sensor provided on the robot.

12. The robot of claim 11, wherein the offboard sensor includes a mobile computing device of the human actor.

13. The robot of claim 12, wherein the mobile computing device of the human actor communicates a velocity of the human actor.

14. The robot of claim 9, wherein detecting the mood of the human actor includes detecting, based on input detected from the multiple onboard sensors, a traffic pattern in the region where the robot operates.

15. A method for controlling a robot, the method comprising:
obtaining, by a processor of a computer system, sensor data from multiple onboard sensors of the robot;
based on input detected from the multiple onboard sensors, determining, by the processor of the computer system, a physical characteristic of a human actor that is located in a region where the robot operates;
detecting, by the processor of the computer system, a mood of the human actor based on the physical characteristic; and
based on the detected mood of the human actor, causing, by the processor of the computer system, the robot to alter its operation.
